(19) United States
(12) Patent Application Publication
(10) Pub. No.: US 2015/0250552 A1
(43) Pub. Date: Sep. 10, 2015

(54) ADVANCED METHODS OF MODELING KNEE JOINT KINEMATICS AND DESIGNING SURGICAL REPAIR SYSTEMS

(71) Applicant: ConforMIS, Inc., Bedford, MA (US)

(72) Inventors: Klaus Radermacher, Aachen (DE); Malte Asseln, Aachen (DE); Ghaith Al Hares, Aachen (DE); Philipp Lang, Lexington, MA (US); Daniel Steines, Lexington, MA (US)

(21) Appl. No.: 14/617,912

(22) Filed: Feb. 9, 2015

Related U.S. Application Data
(60) Provisional application No. 61/937,501, filed on Feb. 8, 2014.

(51) Int. Cl. A61B 19/00; G06F 19/00; A61F 2/46

(52) U.S. Cl. CPC A61B 19/50; A61F 2/46; G06F 19/3437; A61B 2019/505; A61B 2019/508; A61F 2002/4633; A61F 2240/004

(57) ABSTRACT

Various embodiments of selecting and/or designing one or more aspects of patient-adapted surgical repair systems based, at least in part, on implementation of patient-adapted biomotion simulation models are disclosed herein.

### RELATED APPLICATIONS

This application claims the benefit of U.S. Provisional Application No. 61/937,501, entitled “Advanced Methods Of Modeling Knee Joint Kinematics And Designing Surgical Repair Systems,” filed Feb. 8, 2014, which is incorporated herein by reference in its entirety.

### FIELD

The present disclosure generally relates to surgical repair systems (e.g., resection cut strategy, guide tools, and implant components) as described in, for example, U.S. patent application Ser. No. 13/397,457, entitled “Patient-Adapted and Improved Orthopedic Implants, Designs And Related Tools,” filed Feb. 15, 2012, and published as U.S. Patent Publication No. 2012-0209394, which is incorporated herein by reference in its entirety. The present teachings also relate to anatomical models, anatomical simulations, and the design of surgical repair systems as described in, for example, U.S. patent application Ser. No. 14/169,093, entitled “Advanced Methods and Techniques for Designing Knee Implant Components,” filed Jan. 30, 2014, and published as U.S. Patent Publication No. 2014-0222390, which is incorporated herein by reference in its entirety, and International Application No. PCT/US14/30001, entitled “Kinematic and Parameterized Modeling for Patient-Adapted Implants, Tools, And Surgical Procedures,” filed Mar. 15, 2014, published as WO 2014/145267, and which claims priority to U.S. Patent Application Ser. No. 61/801,865, entitled “Modeling, Analyzing and Using Anatomical Data for Patient Adapted Implants, Designs, Tools and Surgical Procedures,” filed Mar. 15, 2013, each of which are incorporated herein by reference in its entirety. Aspects of the present disclosure also relate to methods of acquiring and utilizing patient-specific information as described in, for example, U.S. patent application Ser. No. 14/168,947, entitled “Acquiring and Utilizing Kinematic Information for Patient-Adapted Implants, Tools and Surgical Procedures,” filed Jan. 30, 2014, published as U.S. Patent Publication No. 2014-0222157, which is incorporated herein by reference in its entirety.

### BRIEF DESCRIPTION OF THE DRAWINGS

FIG. 1 is a flow chart illustrating an exemplary process for generating a model of a patient's joint;

FIG. 2a depicts exemplary image data from which edges of a patient's femur and tibia may be identified;

FIG. 2b depicts a 3D representation of the biological structure of a patient's knee joint created from segmented and selected data from multiple images;

FIG. 3 is a flowchart illustrating a procedure for validating patient-specific biomechanical knee model simulations;

FIGS. 4a-c depict personalized biomechanical knee models for each of three individual subjects, respectively;

FIGS. 5a-c are graphs comparing simulated tibiofemoral rotation data with corresponding measured data for each of three subjects, respectively;

FIGS. 6a-c are graphs comparing simulated tibiofemoral medial-lateral translation data with corresponding measured data for each of three subjects, respectively;

FIGS. 7a-c are graphs comparing simulated tibiofemoral anterior-posterior translation data with corresponding measured data for each of three subjects, respectively; and

FIGS. 8a-c are graphs comparing simulated tibiofemoral proximal-distal translation data with corresponding measured data for each of three subjects, respectively.

### DETAILED DESCRIPTION

In this application, the use of the singular includes the plural unless specifically stated otherwise. Furthermore, the use of the term “including,” as well as other forms, such as “includes” and “included,” is not limiting. Also, terms such as “element” or “component” encompass both elements and components comprising one unit and elements and components that comprise more than one subunit, unless specifically stated otherwise. Also, the use of the term “portion” may include part of a moiety or the entire moiety.

The section headings used herein are for organizational purposes only and are not to be construed as limiting the subject matter described.

Various embodiments described herein include the use of automated, and/or semi-automated computing systems to obtain, quantify, classify, model, and/or simulate patient anatomical information for use in selecting and/or designing surgical tools, implants and/or surgical procedures to repair and/or replace portions of a patient's anatomy. The models created can include actual and/or approximate models of the patient's existing anatomy as well as models of optimal, desired, undesired and/or unacceptable anatomy derived using, at least in part, the patient's existing anatomical data. The derived models can be created using a wide variety of tools, techniques and/or data sources.

The image data, derived models and/or actual models, and/or simulations can be utilized to select, design and/or manufacture surgical tools, implants and surgical techniques that, when utilized on the patient, create an optimal and/or otherwise acceptable repair and/or replacement of the relevant patient anatomy. These models will also desirably facilitate the creation of highly durable implant components that can be easily implanted using less invasive and/or least invasive surgical techniques.

In some embodiments, an initial step in repairing and/replacing one or more anatomical features of a patient can be to assess the size, shape and/or condition of the relevant patient anatomy. For an orthopedic implant, this process typically includes obtaining one or more images of the patient's joint and/or other relevant patient anatomy (e.g., adjacent anatomical areas and/or other features of interest) using, for example, non-invasive imaging modalities (as well as other imaging and/or anatomical derivation techniques known in the art). The raw electronic image data can be used to create one or more representations or “models” of the patient's anatomy. These representations can include electronic models as well as 2-Dimensional images and/or 3-Dimensional physical reproductions of the patient anatomy.

In various embodiments, the models can be used to select and/or design an orthopedic implant appropriate for the patient's anatomy. In other embodiments, the models can be processed and/or modified to generate one or more modified versions of the patient anatomy, including portions of a joint and/or surfaces within or adjacent to the joint, with the derived model(s) representing desired (and/or undesired) conditions of the joint at various stages, including after surgical repair and/or replacement. In various embodiments, the raw image data can be used to create models that can be used to analyze the patient's existing joint structure and kinematics, and to devise and evaluate a course of corrective action, including surgical implants, tools, and/or procedures.

In some embodiments, the data and/or models can be used to design an implant having one or more patient-specific features, such as a surface or curvature. Additionally or alternatively, the various models described herein can be utilized to plan a surgical procedure as well as to design and/or select surgical tools useful during the procedure. In various embodiments, the models can be optimized or otherwise modified using a wide variety of techniques and/or data sources, to create one or more desired models that represent one or more desired “improvements” or outcomes of a surgical repair and/or replacement procedure.

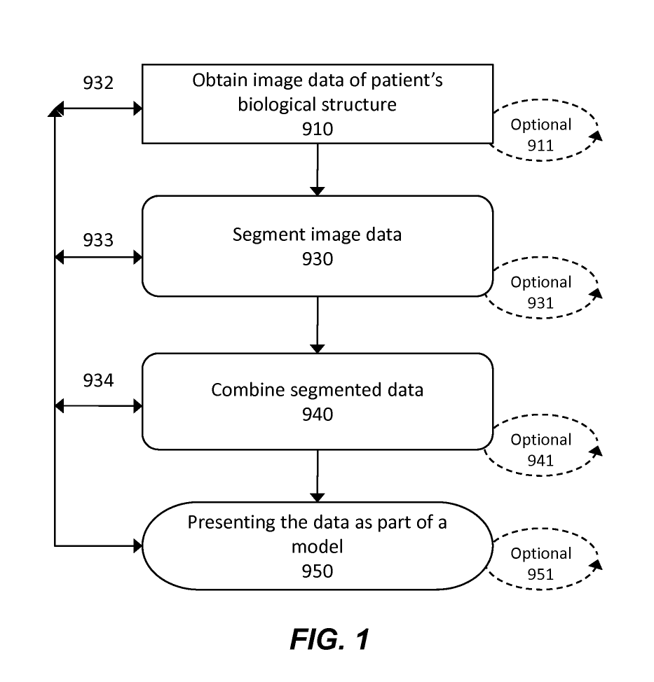

One initial step in many embodiments is to obtain image data of a patient's anatomy. As illustrated in FIG. 1, a method of generating a model of a patient's joint or other biological feature can include one or more of the steps of obtaining image data of a patient's biological structure 910; analyzing or segmenting the image data 930; combining the segmented data 940; and presenting the data as part of a model 950.

Image data can be obtained 910 from near or within the patient's biological structure(s) of interest. For example, pixel or voxel data from one or more radiographic or tomographic images of a patient's joint can be obtained, for example, using computed or magnetic resonance tomography. A wide variety of imaging modalities known in the art can be used, including X-ray, ultrasound, laser imaging, MRI, PET, SPECT, radiography including digital radiography, digital tomosynthesis, cone beam CT, and contrast enhanced imaging. Image data can also include electronic image data derived from physical image “films” or “plates” through scanning or other capture techniques.

The one or more pixels or voxels (as well as other electronic values representing the image data) can be converted into one or a set of values. For example, a single pixel/voxel or a group of pixels/voxels can be converted to coordinate values, e.g., a point in a 2D or 3D coordinate system. The set of values also can include a value corresponding to the pixel/voxel intensity or relative grayscale color. Moreover, the set of values can include information about neighboring pixels or voxels, for example, information corresponding to relative intensity or grayscale color and or information corresponding to relative position.

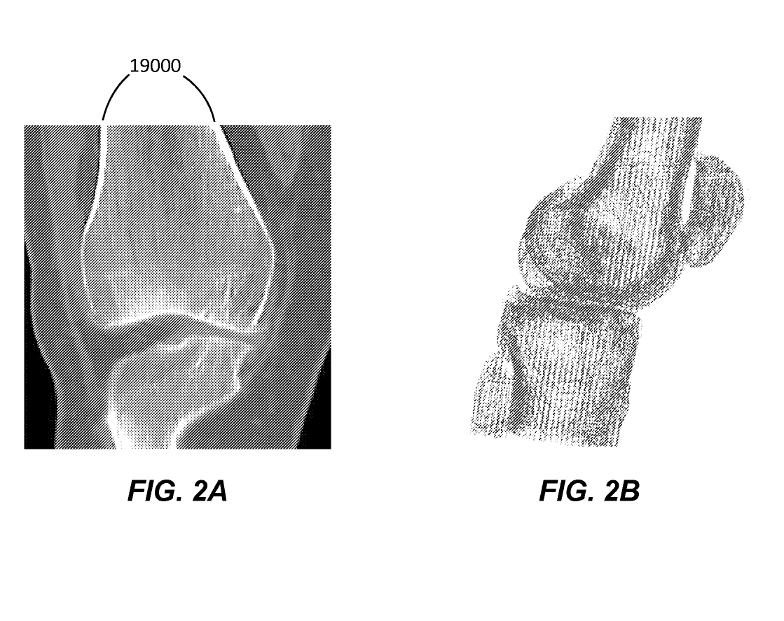

Then, the image data can be analyzed or segmented 930 to identify those data corresponding to a particular biological feature of interest. For example, as shown in FIG. 2a, image data can be used to identify the edges of a biological structure, in this case, the surface outline for each of the patient's femur and tibia. As shown, the distinctive transition in color intensity or grayscale 19000 at the surface of the structure can be used to identify pixels, voxels, corresponding data points, a continuous line, and/or surface data representing the surface or other feature of the biological structure. This step can be performed automatically (for example, by a computer program operator function) or manually (for example, by a clinician or technician), or by a combination of the two.

Optionally, the segmented data can be combined 940. For example, in a single image, segmented and selected reference points (e.g., derived from pixels or voxels) and/or other data can be combined to create one or more lines representing the surface outline of a biological structure. Moreover, as shown in FIG. 2b, the segmented and selected data from multiple images can be combined to create a 3D representation of the biological structure. Alternatively, the images can be combined to form a 3D data set, from which the 3D representation of the biological structure can be derived directly using a 3D segmentation technique, for example an active surface or active shape model algorithm or other model based or surface fitting algorithm.

Optionally, the 3D representation of the biological structure can be generated, manipulated, smoothed and/or corrected, for example, by employing a 3D polygon surface, a subdivision surface or parametric surface such as, for example, a non-uniform rational B-spline (NURBS) surface. Various methods are available for creating a parametric surface. In various embodiments, the 3D representation can be converted directly into a parametric surface by connecting data points to create a surface of polygons and applying rules for polygon curvatures, surface curvatures, and other features. Alternatively, a parametric surface can be best-fit to the 3D representation, for example, using publicly available software such as Geomagic® software (Research Triangle Park, N.C.).

Then, the data can be presented as part of a model 950, for example, a patient-specific virtual model that includes the biological feature(s) of interest. The data can be utilized to create multiple models, representing different anatomical features (i.e., individual models representing bone surfaces, bone structure variations or interfaces, articulating surfaces, muscles and/or connective tissues, the patient's skin surface, etc.) or a single model can incorporate multiple features of interest.

As will be appreciated by those of skill in the art, one or more of these steps 910, 930, 940, 950 can be repeated 911, 931, 941, 951 as often as desired to achieve the desired result. Moreover, the steps can be repeated reiteratively 932, 933, 934. If desired, the practitioner can proceed directly 933 from the step of segmenting image data 930 to presenting the data as part of a model 950.

In various embodiments, individual images of a patient's biological structure can be segmented individually and then, in a later step, the segmentation data from each image can be combined. The images that are segmented individually can be one of a series of images, for example, a series of coronal tomographic slices (e.g., front to back) and/or a series of sagittal tomographic slices (e.g., side to side) and/or a series of axial tomographic slices (e.g., top to bottom) of the patient's joint. In some cases, segmenting each image individually can create noise in the combined segmented data. As an illustrative example, in an independent segmentation process, an alteration in the segmentation of a single image may not alter the segmentation in contiguous images in a series. Accordingly, an individual image can be segmented to show data that appears discontinuous with data from contiguous images. To address this issue, certain embodiments include methods for generating a model from a collection of images, for example, simultaneously, rather than from individually segmented images. One such method is referred to as deformable segmentation, as described in, for example, U.S. Patent Publication No. 2012-0209394.

In various embodiments, information collected from a patient or patient group, including the image data and/or models described herein, can include points, surfaces, and/or landmarks, collectively referred to herein as “reference points.” In certain embodiments, the reference points can be selected and used to derive a varied or altered surface, such as, without limitation, an ideal surface or structure.

In various embodiments, reference points can be used to create a model of the patient's relevant biological feature(s) and/or one or more patient-adapted surgical steps, tools, and implant components. For example the reference points can be used to design a patient-adapted implant component having at least one patient-specific or patient-engineered feature, such as a surface, dimension, or other feature.

Sets of reference points can be grouped to form reference structures used to create a model of a joint, an implant design, and/or a tool design. Designed implant and/or tool surfaces can be derived from single reference points, triangles, polygons, or more complex surfaces, such as parametric or subdivision surfaces, or models of joint material, such as, for example, articular cartilage, subchondral bone, cortical bone, endosteal bone or bone marrow. Various reference points and reference structures can be selected and manipulated to derive a varied or altered surface, such as, without limitation, an ideal surface or structure.

The reference points can be located on or in the joint that will receive the patient-adapted implant. For example, the reference points can include weight-bearing surfaces or locations in or on the joint, a cortex in the joint, cortical and/or cancellous wall boundaries, and/or an endosteal surface of the joint. The reference points also can include surfaces or locations outside of but related to the joint. Specifically, reference points can include surfaces or locations functionally related to the joint.

For example, in embodiments directed to the knee joint, reference points can include one or more locations ranging from the hip down to the ankle or foot. The reference points also can include surfaces or locations homologous to the joint receiving the implant. For example, in embodiments directed to a knee, a hip, or a shoulder joint, reference points can include one or more surfaces or locations from the contralateral knee, hip, or shoulder joint.

Reference points and/or data for obtaining measurements of a patient's joint, for example, relative-position measurements, length or distance measurements, curvature measurements, surface contour measurements, thickness measurements (in one location or across a surface), volume measurements (filled or empty volume), density measurements, and other measurements, can be obtained using any suitable technique. For example, one dimensional, two-dimensional, and/or three-dimensional measurements can be obtained using data collected from mechanical means, laser devices, electromagnetic or optical tracking systems, molds, materials applied to the articular surface that harden as a negative match of the surface contour, and/or one or more imaging techniques described above herein and/or known in the art. Data and measurements can be obtained non-invasively and/or preoperatively. Alternatively, measurements can be obtained intraoperatively, for example, using a probe or other surgical device during surgery.

In certain embodiments, imaging data collected from the patient, for example, imaging data from one or more of x-ray imaging, digital tomosynthesis, cone beam CT, non-spiral or spiral CT, non-isotropic or isotropic MRI, SPECT, PET, ultrasound, laser imaging, and/or photo-acoustic imaging is used to qualitatively and/or quantitatively measure one or more of a patient's biological features, one or more of normal cartilage, diseased cartilage, a cartilage defect, an area of denuded cartilage, subchondral bone, cortical bone, endosteal bone, bone marrow, a ligament, a ligament attachment or origin, menisci, labrum, a joint capsule, articular structures, and/or voids or spaces between or within any of these structures. The qualitatively and/or quantitatively measured biological features can include, but are not limited to, one or more of length, width, height, depth and/or thickness; curvature, for example, curvature in two dimensions (e.g., curvature in or projected onto a plane), curvature in three dimensions, and/or a radius or radii of curvature; shape, for example, two-dimensional shape or three-dimensional shape; area, for example, surface area and/or surface contour; perimeter shape; and/or volume of, for example, the patient's cartilage, bone (subchondral bone, cortical bone, endosteal bone, and/or other bone), ligament, and/or voids or spaces between them. In certain embodiments, measurements of biological features can include any one or more of the illustrative measurements identified in U.S. Patent Publication No. 2012-0209394. Additional patient-specific measurements and information that can be used in the evaluation can include, for example, joint kinematic measurements, bone density measurements, bone porosity measurements, soft and connective tissues structures, skin, muscles, identification of damaged or deformed tissues or structures, and patient information, such as patient age, weight, gender, ethnicity, activity level, and overall health status. Moreover, the patient-specific measurements may be compared, analyzed or otherwise modified based on one or more “normalized” patient model or models, or by reference to a desired database of anatomical features of interest. For example, a series of patient-specific femoral measurements may be compiled and compared to one or more exemplary femoral or tibial measurements from a library or other database of “normal” (or other reference population) femur measurements. Comparisons and analysis thereof may concern, but is not limited to, one or more or any combination of the following dimensions: femoral shape, length, width, height, of one or both condyles, intercondylar shapes and dimensions, trochlea shape and dimensions, coronal curvature, sagittal curvature, cortical/cancellous bone volume and/or quality, etc., and a series of recommendations and/or modifications may be accomplished. Any parameter mentioned throughout the specification, including anatomic, biomechanical and kinematic parameters, can be utilized, not only in the knee, but also in the hip, shoulder, ankle, elbow, wrist, spine and other joints. Such analysis may include modification of one or more patient-specific features and/or design criteria for the implant to account for any underlying deformity reflected in the patient-specific measurements. If desired, the modified data may then be utilized to select and/or design an appropriate implant and/or tool to match the modified features, and a final verification operation may be accomplished to ensure the selected and/or designed implant and/or tool is acceptable and appropriate to the original unmodified patient-specific measurements (i.e., the selected and/or designed implant and/or tool will ultimately “fit” the original patient anatomy). In alternative embodiments, the various anatomical features may be differently “weighted” during the comparison process (utilizing various formulaic weightings and/or mathematical algorithms), based on their relative importance or other criteria chosen by the designer/programmer and/or physician.

In one exemplary embodiment, the various anatomical features of the tibia (i.e., anterior-posterior and/or medial-lateral dimensions, perimeters, medial/lateral slope, shape, tibial spine height, and other features) could be measured, modeled, and then compared to and/or modified based on a database of one or more “normal” or “healthy” tibial measurements and/or models, with the resulting information used to identify anatomical deformities and/or used to select and/or design a desired implant shape, size and placement. If desired, similar verification of implant appropriateness to the original measured parameters may be accomplished as well. In various embodiments, the various anatomical features of any joint can be measured and then compared/modified based on a database of “healthy” or otherwise appropriate measurements of appropriate joints, including those of a medial condyle, a lateral condyle, a trochlea, a medial tibia, a lateral tibia, an entire tibia, a medial patella, a lateral patella, an entire patella, a medial trochlea, a central trochlea, a lateral trochlea, a portion of a femoral head, an entire femoral head, a portion of an acetabulum, an entire acetabulum, a portion of a glenoid, an entire glenoid, a portion of a humeral head, an entire humeral head, a portion of an ankle joint, an entire ankle joint and/or a portion or an entire elbow, wrist, hand, finger, spine, or facet joint.

In addition to (or optionally in place of) the above-mentioned measurements, it may be desirable to obtain measurements of the targeted joint (as well as surrounding anatomical areas and/or other joints of the patient's anatomy) in a weight-bearing condition. In various embodiments, such measurements may be obtained using techniques, such as, for example, those described in U.S. patent application Ser. No. 14/168,947. Such measurements can provide data on the alignment and/or movement of the joint and surrounding structures (as well as the loading conditions of the various joint components)—information which may be difficult to obtain or model from standard imaging techniques (i.e., sitting or lying X-rays, CT-scans and/or MRI imaging). Such load-bearing measurements can include imaging of the patient standing, kneeling, walking and/or carrying loads of varying sizes and/or weights. Weight-bearing data and kinematic information may be used, for example, as input for, modification of, and/or evaluation of biomechanical models/simulations (e.g., as described below) and/or to optimize parameters of patient-adapted surgical repair systems, as discussed herein.

In certain embodiments, a computer program simulating biomotion of one or more joints, such as, for example, a knee joint, or a knee and ankle joint, or a hip, knee and/or ankle joint, can be utilized. In certain embodiments, imaging data as previously described, which can include information related to the joint or extremity of interest as well as information regarding adjacent anatomical structures, can be entered into the computer program. In addition to (or in place of) patient-specific image data, patient-specific kinematic data, for example obtained as described above, can be entered into the computer program. Alternatively, patient-specific navigation data, for example generated using a surgical navigation system, image guided or non-image guided, can be entered into the computer program. This kinematic or navigation data can, for example, be generated by applying optical or RF markers to the limb and by registering the markers and then measuring limb movements, for example, flexion, extension, abduction, adduction, rotation, and other limb movements.

Optionally, other data including anthropometric data may be added for each patient. These data can include but are not limited to the patient's age, gender, weight, height, size, body mass index, and race. Desired limb alignment and/or deformity correction can be added into the model. The position of bone cuts on one or more articular surfaces as well as the intended location of implant bearing surfaces on one or more articular surfaces can be entered into the model.

A patient-specific biomotion model can be derived that includes combinations of parameters discussed above. The biomotion model may simulate various activities of daily life, including normal gait, stair climbing, descending stairs, running, kneeling, squatting, sitting and any other physical activity (including activities relevant to other joints of interest).

In some embodiments, the biomotion model can start out with standardized activities, typically derived from reference databases. These reference databases can be generated, for example, using biomotion measurements using force plates and motion trackers using radiofrequency or optical markers and video equipment. Additionally or alternatively, reference databases can be generated using kinematic measurements, e.g., as discussed above, and/or using averaged information from a plurality of specific biomotion simulations.

The biomotion model can then be individualized with use of patient-specific information including, for example, at least one of, but not limited to, the patient's age, gender, weight, height, body mass index, and race, the desired limb alignment or deformity correction, and the patient's imaging data, for example, a series of two-dimensional images or a three-dimensional representation of the joint for which surgery is contemplated.

In some embodiments, a biomotion simulation model can be implemented and adapted to subject-specific cases in a multi-body simulation software (e.g., AnyBody v6.0, AnyBody Technology A/S, Denmark). For example, for implementation of a biomechanical knee model, the StandingModel from the AnyBody Managed Model Repository 1.5 utilized with various adaptations. A standard hinge joint may be replaced with a complex knee joint, such as, for example, one comprising six degrees of freedom. 3D bone geometries may be obtained via any of a variety of methods, including, for example, one or more of those methods discussed above. By way of example, in some embodiments, 3D bone geometry may be obtained from an optimized MRI scan using manual segmentation (e.g., as described in U.S. patent application Ser. No. 14/168,947) and then may be post-processed by mesh reduction and smoothing filters (e.g., those available in the mesh processing software MeshLab, Visual Computing Lab ISTI-CNR) to reduce the stepping effect associated with the manual segmentation. Further, in some embodiments, a homogenous dilation for each bone may be generated and used as articulating surfaces. For example, in some embodiments, a homogenous dilation of 3 mm may be used as articulating surfaces.

In some embodiments, the biomotion simulation model may further incorporate the anatomical locations of one or more ligaments (e.g., ACL, PCL, MCL, LCL) and muscle attachments. The anatomical locations of one or more ligaments and muscle attachments may be determined according to any of the various methods described elsewhere herein. For example, in some embodiments, such locations may be determined based on literature data. In some embodiments, ligament parameters, such as, for example, elongation and slack length, may be adjusted in a calibration study in a two leg stance as a reference position.

In various embodiments rough overall scaling may be performed for subject-specific adaptation. For example, a general scaling law (e.g., taking segment length, mass and/or fat into account) may be used for a rough overall scaling. In some embodiments, the scaling law may be further modified to allow a detailed adaption of the knee region (e.g., distal femur, patella and proximal tibia). Such detailed adaptation may be utilized to, for example, align the subject-specific knee morphology (optionally, including ligament and muscle attachments) in the reference model.

A variety of boundary conditions may be utilized, depending, for example, the information available. In some embodiments, the boundary conditions may be solely described by analytical methods (e.g., if body motion and/or force data are not available). In some embodiments, ground reaction forces may be predicted by adding muscle forces between the foot and environment which are solved by the AnyBody muscle recruitment optimization process. Further, in some embodiments, a simulation may include one or more kinematic constraints. For example, in some embodiments, a single leg deep knee bend may be simulated such that the center of mass is positioned above the ankle joint. In various embodiments, contact forces in the knee joint may be computed using a force dependent kinematic algorithm, for example, as described in Andersen M. S., et al.: Proceedings of the ISB Conference, 2011, which is incorporated herein by reference in its entirety. In various embodiments, information regarding abduction/adduction movement may also be included/simulated. In some embodiments, the simulation may be adapted to account for other patient-specific factors, such as, for example, gender, age, fitness level, and/or posture.

Aspects of a surgical repair system, such as an implant shape, associated bone cuts generated in various optimizations and/or modifications discussed herein, for example, limb alignment, deformity correction and/or bone preservation on one or more articular surfaces, can be introduced into any of the various embodiments of biomotion simulation models disclosed herein. Based on one or more parameters measured in a patient-specific biomotion model, one or more parameters associated with the surgical repair system may be optimized and/or modified. Table 1 includes an exemplary list of parameters that can be measured in a patient-specific biomotion model.

TABLE 1
Parameters measured in a patient-specific biomotion model.

| Joint implant | Measured Parameter |
|---|---|
| knee | Medial femoral rollback during flexion |
| knee | Lateral femoral rollback during flexion |
| knee | Patellar position, medial, lateral, superior, inferior for |
| | different flexion and extension angles |
| knee | Internal and external rotation of one or more femoral condyles |
| knee | Internal and external rotation of the tibia |
| knee | Flexion and extension angles of one or more articular surfaces |
| knee | Anterior slide and posterior slide of at least one of the medial |
| | and lateral femoral condyles during flexion or extension |
| knee | Medial and lateral laxity throughout the range of motion |
| knee | Contact pressure or forces on at least one or more articular |
| | surfaces e.g., a femoral condyle and a tibial plateau, a trochlea |
| | and a patella |
| knee | Contact area on at least one or more articular surfaces, e.g., a |
| | femoral condyle and a tibial plateau, a trochlea and a patella |
| knee | Forces between the bone-facing surface of the implant an |
| | optional cement interface and the adjacent bone or bone |
| | marrow, measured at least one or multiple bone cut or bone- |
| | facing surface of the implant on at least one or multiple |
| | articular surfaces or implant components. |
| knee | Ligament location, e.g., ACL, PCL, MCL, LCL, retinacula, |
| | joint capsule, estimated or derived, for example using an |
| | imaging test. |
| knee | Ligament tension, strain, shear force, estimated failure forces, |
| | loads for example for different angles of flexion, extension, |
| | rotation, abduction, adduction, with the different positions or |
| | movements optionally simulated in a virtual environment. |
| knee | Potential implant impingement on other articular structures, |
| | e.g., in high flexion, high extension, internal or external |
| | rotation, abduction or adduction or any combinations thereof |
| | or other angles/positions/movements. |

The above list is not meant to be exhaustive, but only exemplary. Any other biomechanical parameter known in the art can be included in the analysis.

The information from the measurements and/or models described above can then be utilized (alone or in combination with other data described herein) to design and/or modify various features of a joint repair system. The implant, instrument, and/or procedure design may be optimized with the objective to establish normal or near normal kinematics. The implant optimizations can include one or multiple implant components. Implant and/or procedure optimizations based on patient-specific data include (but are not limited to):
Changes to external, joint-facing implant shape in coronal plane
Changes to external, joint-facing implant shape in sagittal plane
Changes to external, joint-facing implant shape in axial plane
Changes to external, joint-facing implant shape in multiple planes or three dimensions
Changes to internal, bone-facing implant shape in coronal plane
Changes to internal, bone-facing implant shape in sagittal plane
Changes to internal, bone-facing implant shape in axial plane
Changes to internal, bone-facing implant shape in multiple planes or three dimensions
Changes to one or more bone cuts, for example with regard to depth of cut, orientation of cut, joint-line location, and/or joint gap width

When changes are made on multiple articular surfaces or implant components, these can be made in reference to or linked to each other. For example, in the knee, a change made to a femoral bone cut based on patient-specific data can be referenced to or linked with a concomitant change to a bone cut on an opposing tibial surface, for example, if less femoral bone is resected, more tibial bone may be resected.

### Example Biomotion Simulation Model

A biomotion simulation model, as described above, was developed and adapted to three subjects. In particular, the StandingModel from the AnyBody Managed Model Repository 1.5 was utilized, with a complex knee joint having six degrees of freedom. 3D bone geometry were obtained from an optimized MRI scan using manual segmentation as described in Al Hares, G., In: Proceedings of the 13th annual meeting of CAOS international, pp. 197-199, 2013 (which is incorporated herein by reference in its entirety) and then post-processed by mesh reduction and smoothing filters in the mesh processing software MeshLab, Visual Computing Lab ISTI-CNR. A homogenous dilation of 3 mm was used as articulating surfaces. The anatomical locations of the ligaments (ACL, PCL, MCL, LCL) and muscle attachments were determined based on literature data. Ligament parameters were adjusted in a calibration study in a two leg stance as a reference position. For subject-specific adaptation, a general scaling law, taking segment length, mass and fat into account, was used. The scaling law was further modified to allow a detailed adaption of the knee region (distal femur, patella and proximal tibia), e.g., to align the subject-specific knee morphology (including ligament and muscle attachments) in the reference model. The boundary conditions were solely described by analytical methods. Ground reaction forces were predicted by adding muscle forces between the foot and environment, which were solved by the AnyBody muscle recruitment optimization process. A single leg deep knee bend was simulated by kinematic constraints, such as that the center of mass is positioned above the ankle joint. The contact forces in the knee joint were computed using the force dependent kinematic algorithm (Andersen M. S., et al.: Proceedings of the ISB Conference, 2011).

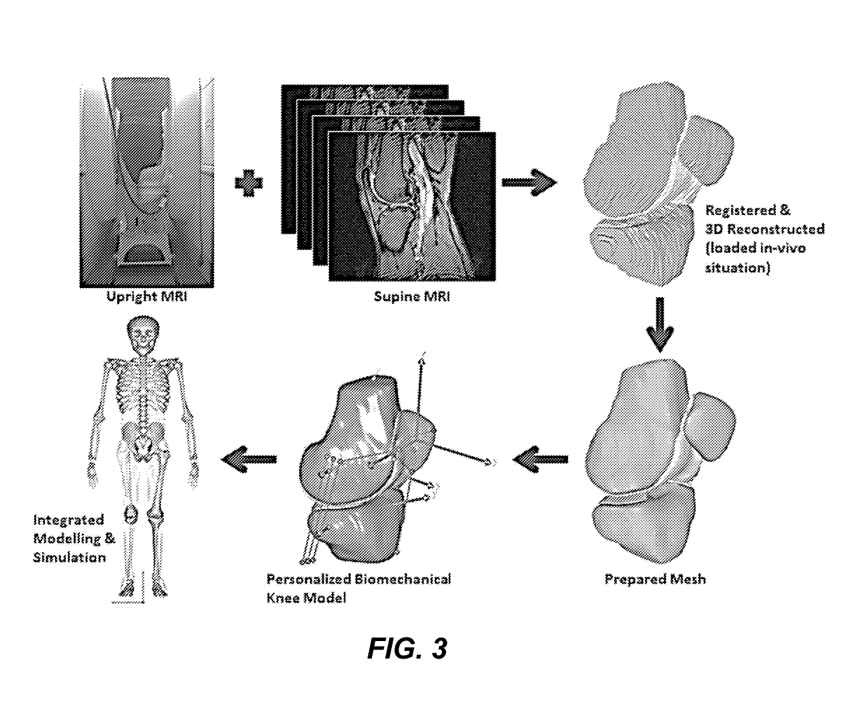

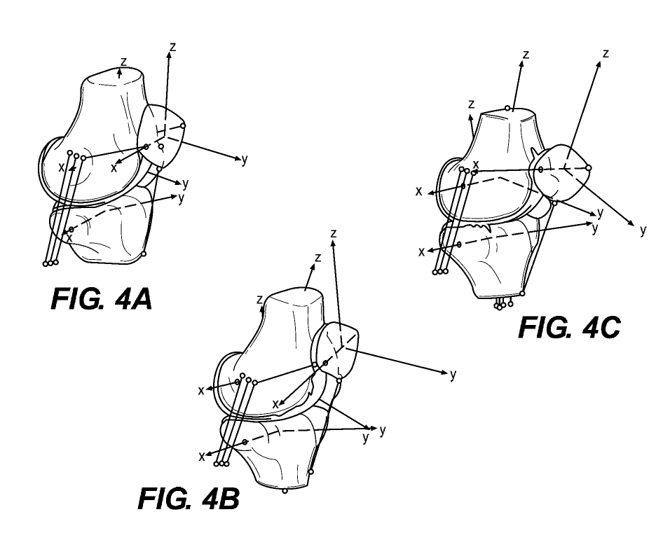

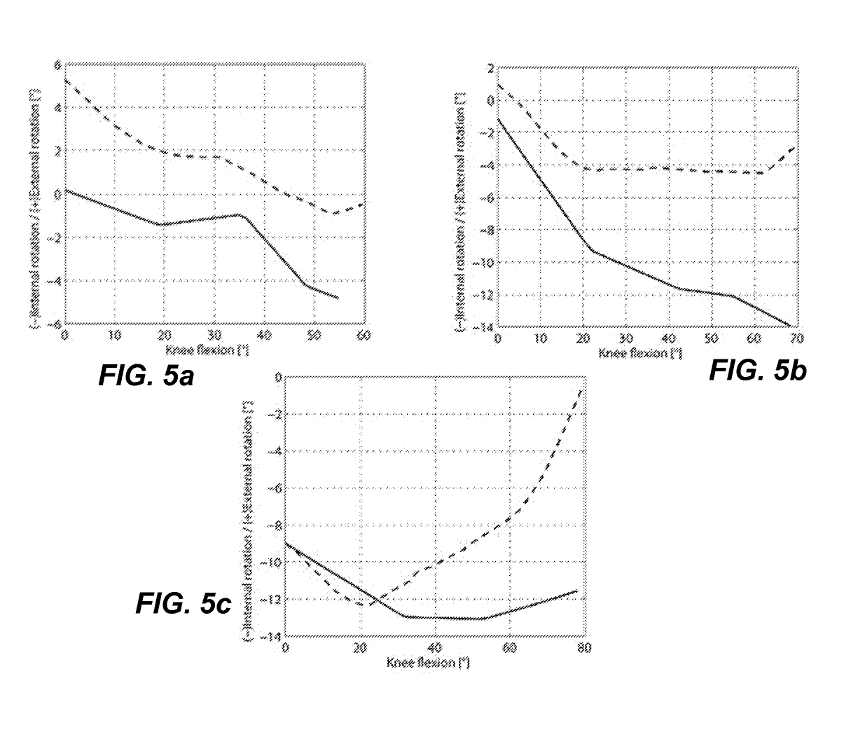

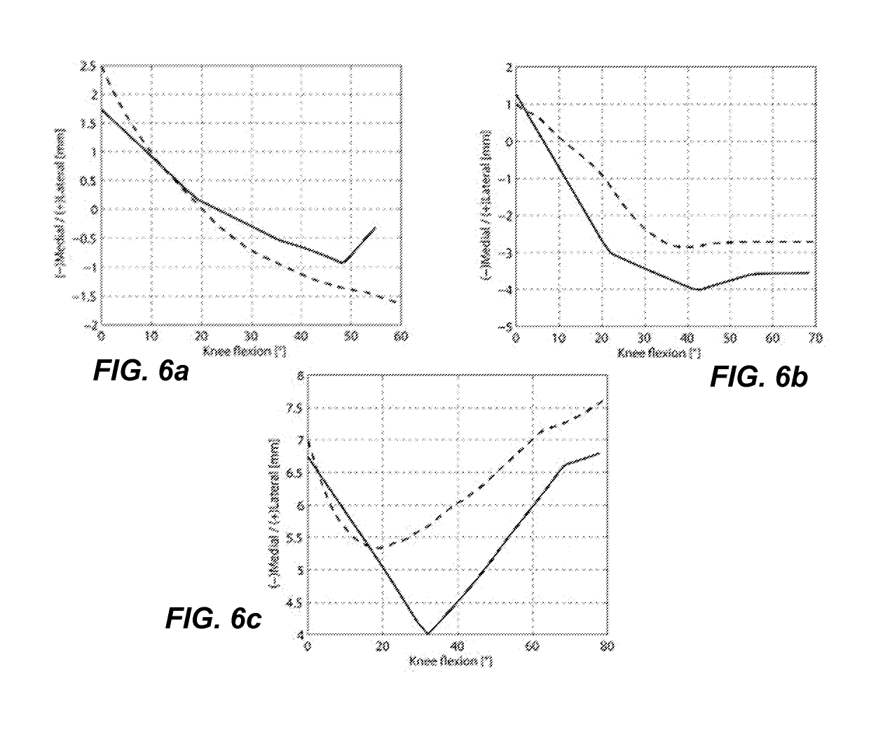

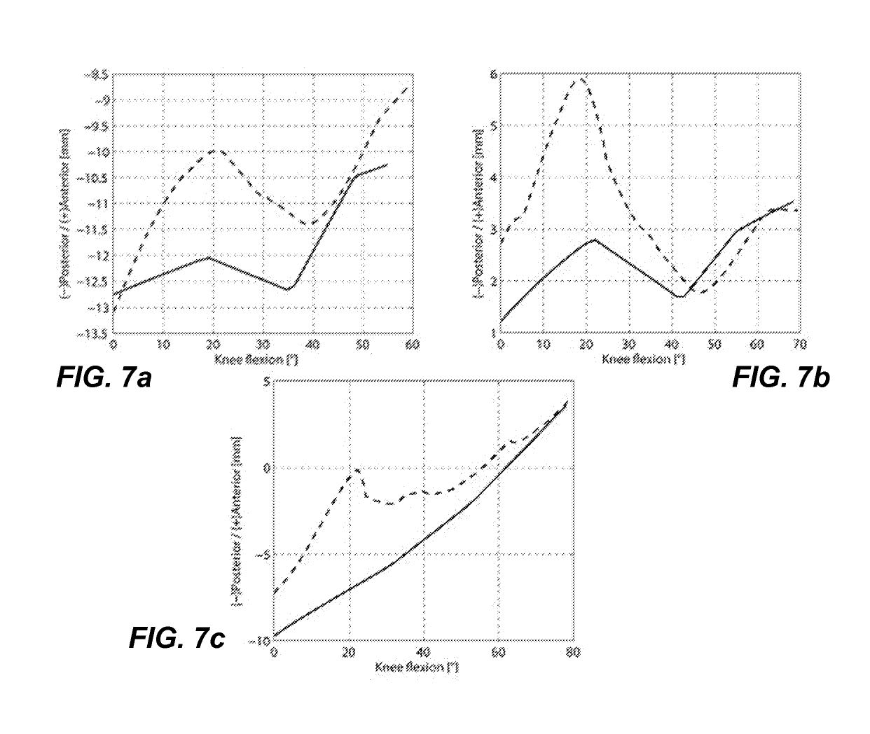

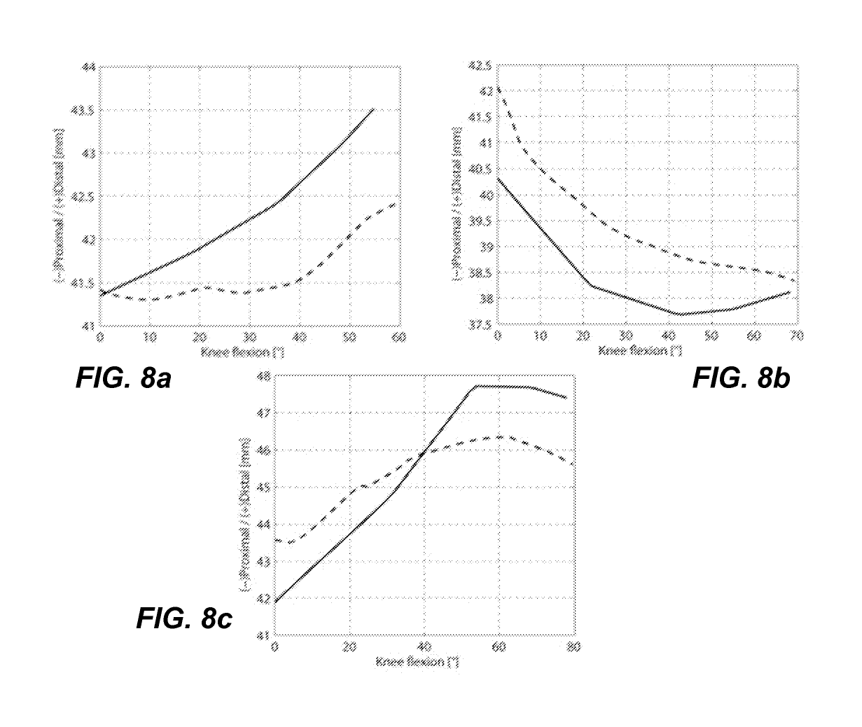

A single leg deep knee bend was simulated, and subject-specific kinematics were recorded, as defined by Grood E S, et al. (J Biomech, 105:136-144, 1983, which is incorporated herein by reference in its entirety). For validation, the simulated kinematic results were then compared to their corresponding subject-specific in-vivo kinematic measurement data obtained under the same full-weight bearing condition, as described in Al Hares, G., In: Proceedings of the 13th annual meeting of CAOS international, pp. 197-199, 2013. FIG. 3 illustrates the workflow for this validation procedure. The whole group of subjects was able to be simulated over the complete range of motion. FIGS. 4a-c depict the personalized biomechanical knee models for subjects 1, 2, and 3, respectively. Graphs of data obtained from the biomotion simulation compared to the corresponding measured data are provided in FIGS. 5-8. FIGS. 5a-c compare simulated tibiofemoral rotation data with corresponding measured data for subjects 1, 2, and 3, respectively. FIGS. 6a-c compare simulated tibiofemoral medial-lateral translation data with corresponding measured data for subjects 1, 2, and 3, respectively. FIGS. 7a-c compare simulated tibiofemoral anterior-posterior translation data with corresponding measured data for subjects 1, 2, and 3, respectively. FIGS. 8a-c compare simulated tibiofemoral proximal-distal translation data with corresponding measured data for subjects 1, 2, and 3, respectively. As can be seen, the tibiofemoral kinematics of three subjects was able to be simulated and predicted the overall trend correctly, while absolute values partially differed.

Thus, this exemplary simulation model, which is highly adaptable to an individual situation, can be suitable to predict, or at least approximate, subject-specific knee kinematics without consideration of cartilage and menisci. This model can enable sensitivity analyses regarding changes in patient specific knee kinematics following, e.g., surgical interventions on bone or soft tissue as well as related to the design and placement of partial or total knee joint replacement components. Accordingly, such a model can be incorporated in the design process of a surgical repair system, including patient-adapted surgical repair systems.

What is claimed is:

1. A method of making a patient-adapted implant for a knee joint of a patient, the method comprising:
obtaining a 3D bone geometry of at least a portion of the joint;
deriving at least a portion of one or more articular surfaces of the joint utilizing a homogenous dilation of the 3D bone geometry;
determining an approximate location of one or more ligament attachments of the joint;
implementing a biomotion simulation model of the joint utilizing the at least a portion of the one or more articular surfaces of the joint and utilizing the approximate location of the one or more ligament attachments;
deriving at least one parameter associated with the joint based, at least in part, on information obtained from the implementing of the biomotion simulation model; and
manufacturing a patient-adapted implant for treating the joint such that the patient-adapted implant includes at least one aspect based, at least in part, on the derived at least one parameter.

2. The method of claim 1, wherein the at least one parameter comprises rollback of a medial portion of a femur of the joint during flexion.

3. The method of claim 1, wherein the at least one parameter comprises rollback of a lateral portion of a femur of the joint during flexion.

4. The method of claim 1, wherein the at least one parameter comprises one or more locations of at least a portion of a patella of the joint at one or more, respective, flexion and/or extension angles of the joint.

5. The method of claim 1, wherein the at least one parameter comprises a degree of internal and/or external rotation of one or more condyles of a femur of the joint.

6. The method of claim 1, wherein the at least one parameter comprises a degree of internal and/or external rotation of at least a portion of a tibia of the joint.

7. The method of claim 1, wherein the homogenous dilation comprises a dilation of about 3 mm.

8. The method of claim 1, wherein the implementing the biomotion simulation model comprises simulating a deep knee bend of the joint.

9. The method of claim 1, wherein the least one aspect comprises a curvature of at least a portion of a joint-facing surface of the implant.

10. A method of making a patient-adapted surgical instrument for treating a knee joint of a patient, the method comprising:
obtaining a 3D bone geometry of at least a portion of the joint;
deriving at least a portion of one or more articular surfaces of the joint utilizing a homogenous dilation of the 3D bone geometry;
determining an approximate location of one or more ligament attachments of the joint;
implementing a biomotion simulation model of the joint utilizing the at least a portion of the one or more articular surfaces of the joint and utilizing the approximate location of the one or more ligament attachments;
deriving at least one parameter associated with the joint based, at least in part, on information obtained from the implementing of the biomotion simulation model; and
manufacturing a patient-adapted surgical instrument for treating the joint such that the patient-adapted surgical instrument includes at least one aspect based, at least in part, on the derived at least one parameter.

11. The method of claim 10, wherein the at least one parameter comprises rollback of a medial portion of a femur of the joint during flexion.

12. The method of claim 10, wherein the at least one parameter comprises rollback of a lateral portion of a femur of the joint during flexion.

13. The method of claim 10, wherein the at least one parameter comprises one or more locations of at least a portion of a patella of the joint at one or more, respective, flexion and/or extension angles of the joint.

14. The method of claim 10, wherein the at least one parameter comprises a degree of internal and/or external rotation of one or more condyles of a femur of the joint.

15. The method of claim 10, wherein the at least one parameter comprises a degree of internal and/or external rotation of at least a portion of a tibia of the joint.

16. The method of claim 10, wherein the homogenous dilation comprises a dilation of about 3 mm.

17. The method of claim 10, wherein the implementing the biomotion simulation model comprises simulating a deep knee bend of the joint.

18. The method of claim 10, wherein the at least one aspect comprises a predetermined depth of a bone cut to be guided by the patient-adapted surgical instrument.
